(19) United States
(12) Patent Application Publication
(10) Pub. No.: US 2010/0137161 A1
(43) Pub. Date: Jun. 3, 2010

(54) METHOD OF GENERATING A PLATELET REACTIVITY PROFILE FOR AN INDIVIDUAL

(75) Inventors: Aaron Peace, Dublin (IE); Dermot Kenny, Dublin (IE)

Correspondence Address: OCCHIUTI ROHLICEK & TSAO, LLP, 10 FAWCETT STREET, CAMBRIDGE, MA, 02138, US

(73) Assignee: ROYAL COLLEGE OF SURGEONS IN IRELAND, Dublin (IE)

(21) Appl. No.: 12/444,480

(22) PCT Filed: Oct. 8, 2007
(86) PCT No.: PCT/IE07/00096

§ 371 Date: Jan. 20, 2010

(30) Foreign Application Priority Data
Oct. 6, 2006 (EP) 06394020.9

(51) Int. Cl. C40B 30/06; C12Q 1/02

(52) U.S. Cl. 506/10; 435/29

(57) ABSTRACT

A method of generating a platelet reactivity profile of an individual comprises the steps of providing a platelet-containing biological sample from the individual, providing at least three platelet function modulators, each platelet function modulator being provided in at least three concentrations, and reacting an aliquot of the platelet containing sample with each concentration of each platelet function modulator in a separate reaction vessel. Platelet aggregation is then measured in each reaction vessel, and the platelet aggregation measurements are used to generate a dose response curve for each platelet function modulator, wherein the dose response curves obtained and/or one or more functions of the dose response curves obtained, comprise a platelet reactivity profile for the individual. Clinical applications of, and kits for carrying out, the methods of the invention are also described.

### TECHNICAL FIELD

The invention relates to methods and kits for generating a platelet reactivity profile for an individual, and clinical and research applications of the platelet reactivity profile.

### BACKGROUND TO THE INVENTION

Cardiovascular disease remains the leading cause of mortality in Europe and the USA. The conundrum remains as to who will suffer from either a heart attack or stroke. Multiple risk prediction models have been formulated to try to succeed in predicting the high risk patients in the population, with some success. Cardiovascular events occur as a result of thrombosis. Thrombosis is the act of clot formation and occurs as a result of platelet activation. This is prevented in part by aspirin, however events still occur. This suggests that the “stickiness” or level of activation between individuals differs and the response to therapy differs also. It is surprising that to this day no one has a certain idea as to what constitutes normal platelet function. This is largely due to the limitations in working with platelets. Unfortunately platelets only survive for approximately 4-6 hours after leaving the body. The process of preparing ex vivo platelets shortens this duration and so only a small number of tests can be performed by an individual, hence providing only a small amount of information on platelet function.

There a small number of commercially available platelet function analysers on the market which have some limitations. The PFA (platelet function analyzer)-100 device measures time to clotting after exposing whole blood to collagen and epinephrine or collagen and ADP known as closure time. The parameter being measured is the closure time. The gold standard test for platelet function is Light Transmission Standard Aggregometry. When one compares PFA-100™ to the gold standard platelet function test the results differ highlighting a major limitation of the device. Unfortunately the Standard aggregometer is limited in that the procedure takes a considerable amount of time to even do a small number of channels as the device tends to have only 4 channels. Accumetrics Verify Now™ device is another device which is used at the bedside, using whole blood to assess platelet function. This device allows one to assess response to Aspirin and Clopidogrel. This test like the PFA-100™ device is limited in that it provides only a limited amount of information regarding platelet reactivity. Further, all of these tests only examine the effects of agonist at a single concentration.

The assessment of platelet reactivity or the ability of the platelet to activate differs between individuals and varies within the same individual at varying time points. Assessing this variability using the currently available tests is difficult, time inefficient and expensive.

It is an object of the invention to overcome at least one of the above problems.

### STATEMENTS OF INVENTION

According to the invention, there is provided a method of generating a platelet reactivity profile of an individual comprising the steps of:
providing a platelet-containing biological sample from the individual;
providing at least three platelet function modulators, each platelet function modulator being provided in at least three concentrations;
reacting an aliquot of the platelet containing sample with each concentration of each platelet function modulator in a separate reaction vessel;
measuring platelet aggregation in each reaction vessel; and
using the platelet aggregation measurements to generate a dose response curve for each platelet function modulator, wherein the dose response curves obtained and/or one or more functions of the dose response curves obtained, comprise a platelet reactivity profile for the individual.

Typically, at least four platelet function modulators are employed. Preferably, at least five platelet function modulators are employed. Ideally, more than five platelet function modulators are employed.

Suitably, the platelet function modulators are either platelet agonists or platelet antagonists. Ideally, the platelet function modulators are platelet agonists.

In one embodiment, the platelet function modulators are selected from the group comprising: TRAP; collagen; epinephrine; ADP; arachidonic acid; serotonin; and thromboxane A2; ristocetin; a NO donor (such as SNAP); U46619; and Convulxin. Typically, the platelet function modulators are selected from the group comprising: TRAP; collagen; epinephrine; ADP; and arachidonic acid.

When TRAP is employed, it is preferable to add TRAP to the reaction vessel after all the other platelet function modulators have been added. Ideally, the TRAP is added to the vessel immediately prior to addition of the platelet-containing sample.

Suitable platelet antagonists will be well known to a person skilled in the field of platelet biology.

Ideally, at least five platelet agonists are employed, the five platelet function modulators being TRAP, collagen, epinephrine, ADP, and arachidonic acid.

In one embodiment, the platelet function profile comprises a dose response curve for each platelet function modulator assayed, in combination with one or more function(s) of the dose response curves, such as hill slope variability, maximum and/or minimum aggregation, and EC values (i.e. EC₅₀). In one embodiment, the prlatelet function profile cosists of only dose response curves, or only EC values.

Suitably, platelet aggregation is determined using light aggregometry. Typically, the light aggregometer is operatively connected to a processor to record and process the readings provided by the light agrregometer. Typically, the processor will include software for processing the data obtained to provide dose response curves for each agonist and, optionally, characteristics of each dose response curve such as hill slope variability and EC values. In a preferred embodiment, the software is GRPAHAD PRISM software.

Typically, the platelet function modulator is provided in at least four, five, six, seven, eight, or nine concentrations. Typically, arachidonic acid will be provided at concentrations that span the range of 1 to 100 mg/ml, preferably span the range of 3 to 70 mg/ml, and more preferably span the range of 0.58 mg/ml to 50 mg/ml. Typically, collagen will be provided that span the range of 0.001 to 0.3 mg/ml, preferably span the range of 0.0015 to 0.25 mg/ml, and more preferably span the range of 0.0023 to 0.19 mg/ml. Typically, ADP will be provided in concentrations that span the range of 0.01 to 40 μM, preferably 0.2 to 30 μM, and more preferably 0.015 to 20 μM. Typically, epinephrine will be provided in concentrations that span the range of 0.01 to 40 μM, preferably 0.125 to 30 μM, and more preferably 0.0122 to 20 μM. Typically, TRAP will be provided in concentrations that span the range of 0.015 to 20 μM. Generally, the range of concentration employed for the platelet function modulators should span four log units, and should encompass the known effective dose range for each of the platelet function modulators employed. The term “span the range” is intended to mean that the concentrations employed range from a lowest value equal to, or adjacent to, the lowest in the range provided above, and a highest value equal to, or adjacent to, the highest value in the range above, with the intermediate concentrations being spread between the extremities.

In a preferred embodiment, the parameter of platelet aggregation measured is maximal aggregation obtained over a period at least 15 minutes, 16 minutes, 17 minutes, and suitably at least 17.5 minutes. Ideally, maximal aggregation obtained is determined over a period of at least 18 minutes.

Suitably, the reaction vessels are wells of a microtitre plate or an equivalent device having a multiplicity of reaction wells. Other types of equivalent devices having such a multiplicity of reaction wells would be known to the person skilled in the art, for example miniaturised microtitre plates, cartridges and the like.

Ideally, the method of the invention is a high-throughput method in which the parameter of platelet aggregation in each reaction vessel is measured substantially simultaneously.

Suitably, each platelet function modulator is provided in at least four, five, six or seven concentrations, preferably spanning at least two log units. Ideally, the platelet function modulator is provided in eight concentrations.

In one embodiment, epinephrine is one of the platelet function modulators, wherein the concentrations of epinephrine employed range from Log [conc] of −8 to Log [conc] of −5. Ideally, the concentrations of epinephrine employed range from Log [conc] of −9 to Log [conc] of −5.

In one embodiment, collagen is one of the platelet function modulators, wherein the concentrations of collagen employed range from Log [conc] of −5.5 to Log [conc] of −3.8. Ideally, the concentrations of collagen employed range from Log [conc] of −5.7 to Log [conc] of −3.7.

In one embodiment, arachidonic acid is one of the platelet function modulators, wherein the concentrations of arachidonic acid employed range from Log [conc] of −5.0 to Log [conc] of −3.4. Ideally, the concentrations of arachidonic acid employed range from Log [conc] of −5.2 to Log [conc] of −3.3.

In a preferred embodiment of the invention, the platelet function modulators are platelet agonists, and wherein at least one of the platelet agonists is employed in a concentration range that includes sub-maximal concentrations for that agonist. Suitably, at least two of the platelet agonists are employed in a concentration range that includes sub-maximal concentrations for that agonist. Preferably, at least three of the platelet agonists are employed in a concentration range that includes sub-maximal concentrations for that agonist. Ideally, the platelet agonists that are employed in a concentration range that includes sub-maximal concentrations for that agonist are one or more of collagen, epinephrine, and arachidonic acid. In this specification, the term “sub-maximal concentration” means the concentration of platelet agonist which induces a degree of aggregation that is less than the concentration of agonist that induces the maximal or greatest response as defined by aggregation.

In this specification, the term “platelet function” refers to the activation status of the platelet sample, i.e. the ability of the platelet to aggregate with other platelets. Suitably, aggregation of platelets is determined by light transmission aggregometry. In this method, light passing through the well containing the reaction mixture is measured prior to and after addition of the platelet function modulator. Thus, when a platelet agonist is added, the absorbance of light passing through the reaction mixture will generally increase due to aggregation of the activated platelets in the well. Generally, light absorbance is measured using a wavelength of between 550 and 590 nm, preferably between 560 and 580 nm and more preferably between 570 and 575 nm. Ideally, light absorbance is measured using a wavelength of 572 nm. Light transmission readers suitable for taking light readings from multiple plates will be well known to those skilled in the art. Further, light absorbance may be measured at different wavelengths such as, for example, 405 nm, 490 nm, and other wavelengths.

The platelet-containing sample is generally selected form the group comprising: washed platelet preparations; and platelet rich plasma (PRP). Suitably, the concentration of platelets in the platelet-containing sample is greater than 2×10⁵/μl, typically greater than 3×10⁵/μl, preferably greater than 4×10⁵/μl, and ideally greater than 5×10⁵/μl. Ideally, the PRP is obtainable by aspirating blood into a solution of citrate, typically a solution of sodium citrate, and generally in a ratio of 10:1 blood:citrate. Typically, the citrate solution has a concentration of between 3% and 4%, ideally 3.2% (w/v). Typically, the volume of agonists/platelet-containing sample (including buffer) added to each well is between 150 and 250 μl, preferably between 180 and 220 μl, most preferably between 195 and 205 μl, and ideally about 200 μl.

Typically, the level of aggregation in each reaction vessel is measured at different time points following the reaction of the platelets with the platelet function modulator. Suitably, at least 2, 3, 4, 5, 6, 7, 8, 9, 10, 12, or 15 readings will be taken during the first 30 minutes, preferably during the first 25 minutes, and most preferably during the first 20 minutes of the reaction. Suitably, readings are taken every 2 to 5 minutes. Ideally, readings are taken at 0, 3, 9, 15, and 18 minutes. Ideally, the plate is agitated between readings, typically employing an orbital shaker. Ideally, the orbital shaker operates at an orbital shaking pattern of between 0.5 and 3 mm, preferably about 1 mm.

The invention also relates to a method of investigating the effects of a test compound on a platelet reactivity profile, which method employs the method of determining the platelet reactivity profile of the invention, wherein the reaction between the agonist and the platelet containing sample is carried out in the presence of the compound. Suitably, the ability of the test compound to agonise or antagonise platelet aggregation is determined by comparing the platelet reactivity profile of a test subject (or subjects) obtained with and without the test compound. Thus, the method of the invention may be employed to test compounds to determine if they are agonists or antagonists of platelet aggregation, to test which biological pathways the compound modulates, and to determine what the optimal concentrations for the compound are. Furthermore, as the method of the invention provides a rapid, high-throughput assay, a library of compounds may be assayed in a rapid and expedient manner.

The invention also relates to a method of determining the platelet reactivity status of an individual which comprises the step of determining the platelet reactivity profile for the individual according to a method of the invention, and comparing the platelet reactivity profile obtained with a reference platelet reactivity profile for that individual. Thus, the method of the invention may be employed to determine whether an individuals platelets have an activation status greater than, or lower that, a reference activation status for that individual. If the individuals platelet reactivity status is greater than a reference for that individual, then the individual may be at risk of suffering an atherothrombotic event. As such, platelet reactivity status may function as a biomarker of the cardiovascular health of the individual, or as an indication as to whether the individual can undergo surgery without a risk of bleeding, or as an indication as to whether the individuals platelets are suitable for transplanting.

The invention also relates to a method of identifying an individual at risk of having an atherothrombotic event, which method comprises the steps of determining the platelet reactivity profile for an individual according to the invention, and comparing the platelet reactivity profile obtained with a reference platelet reactivity profile for that individual, wherein if the platelet reactivity profile for the individual shows a significantly increased response to an agonist as compared to the response to that agonist in the reference platelet reactivity profile, then that individual is at risk of having an atherothrombotic event.

The invention also relates to a method of identifying aberrant platelet reactivity in an individual, which method comprises the steps of determining the platelet reactivity profile for an individual according to a method of the invention, and comparing the platelet reactivity profile obtained with a reference platelet reactivity profile for that individual, wherein if the platelet reactivity profile for the individual is significantly different to the reference platelet reactivity profile, then that individual has aberrant platelet reactivity.

The invention also relates to a method of identifying a suitable anti-platelet agent or dose for an individual in need thereof, which method comprises the steps of determining the platelet reactivity profile for the individual according to a method of the invention, comparing the platelet reactivity profile obtained with a reference platelet reactivity profile for that individual, identifying any agonist for which there is a significantly increased response when compared to the response to that agonist in the reference platelet reactivity profile, and choosing an anti-platelet therapy or dose to target the biological pathway modulated by that agonist.

In the above methods, the reference platelet reactivity profile for the individual depends on the clinical status of the individual. Thus, if the individual under study is a male that is not undergoing anti-platelet therapy, the reference platelet reactivity profile will be an average profile obtained from normal healthy males. However, if the individual under study is undergoing therapeutic intervention, then the reference platelet reactivity profile will be an average profile obtained from patients undergoing the same therapeutic intervention. Generally, in the case of anti-platelet therapy, the treatment will be dual aspirin/clopidogrel. Thus, for example, if the individual is being treated with aspirin/clopidogrel and their platelet reactivity profile indicates that they remain hyper-reactive to TRAP, the a clinician may decide to alter either the dosage of one or both of the drugs, or indeed may decide to change the therapy to specifically target the biological pathway modulated by TRAP (i.e. target the PAR-1 receptor).

Generally, if the method of the invention indicated that a specific agonist is not being adequately inhibited, then a clinician may prescribe a drug that targets the pathway modulated by that agonist. Thus, if the profile indicates inadequate inhibition to TRAP, a drug that targets the PAR1 receptor may be prescribed. Likewise, if the profile indicates inadequate inhibition to collagen or epinbephrine, a Ilbila inhibitor may be prescribed. Likewise, if the profile indicates inadequate inhibition to ADP, CLOPIDOGREL, TICLOPIDINE or PRASUGREL may be prescribed. Likewise, if the profile indicates inadequate inhibition to arachidonic acid, ASPIRIN or a IIbIIIa inhibitor may be prescribed.

Thus, the invention also relates to a method of identifying and correcting inadequate or sub-optimal anti-platelet therapy in an individual, which method comprises the steps of determining the platelet reactivity profile for the individual according to a method of the invention, comparing the platelet reactivity profile obtained with a reference platelet reactivity profile for an individual undergoing the anti-platelet therapy, identifying any agonist which is inadequately inhibited compared with the reference profile, and modifying the anti-platelet therapy to effect adequate inhibition of the agonist. Modification of the therapy may involve changing the drugs employed, or changing the dosage regime for that drug. Thus, for example, if the platelet reactivity profile shows that arachidonic acid induced aggregation is inadequately inhibited compared to the reference platelet reactivity profile, then a clinician may decide to put the patient on aspirin therapy or, if the patient is already on aspirin therapy, change the therapy to increase the dose or prescribe a more effective for of aspirin.

The invention also relates to a method of identifying platelet activity modulating agents, the method comprising the step of determining the platelet reactivity profile for the individual according to a method of the invention in the absence and presence of a test compound, comparing the platelet reactivity profiles obtained, and where thee is a significant difference between the platelet reactivity profiles obtained, determining whether the test compound is a platelet agonist or a platelet antagonist. In a preferred embodiment of the invention, a platelet reactivity profile is obtained for a number of different concentrations of the test compound. The invention also relates to a method of screening a library of test compounds which employ a method of identifying platelet activity modulating agents according to the invention.

In another embodiment, the invention relates to a method of assisting in determining a clinical status of an individual comprising the steps of determining the platelet function profile of the individual according to the method of the invention, and comparing the reactivity profile thus obtained with a reference platelet function profile for that individual, whereby any differences between the test profile and reference profile provides assistance in determining a clinical status of the individual. The clinical status of the individual may include drug responsiveness, drug resistance, surgical risk, cardiovascular risk status, platelet transfusion risk. Drug responsiveness clinical status may include responsiveness to anti-thrombosis agents, such as those selected from the group comprising: aspirin-related drugs; ADP-receptor inhibiting drugs; and GPIIb/IIIa antagonists/blockers.

Thus, the methods of the invention may be used to quickly determine the most suitable anti-thrombosis therapy for an individual, given their platelet function profile. Further, the methods may be used to determine whether an individual is at risk of bleeding during surgery. In one embodiment, the reference platelet function profile may be a profile of a normal healthy male or female donor, in which case correlation will indicate whether the platelet function profile of the patients sample is indicative of normal healthy platelet function. However, if for example the EC₅₀ values obtained from the patient sample are significantly different (i.e. lower) from those of the normal sex-matched reference, then this could indicate that the patients platelets are hyper-responsive thereby predisposing the patient thrombotic events. In this regard, the methods of the invention provide a method of prognosis of cardiovascular morbidity and/or mortality.

The methods of the invention generally involve generating a a platelet reactivity profile for an individual, and comparing that reactivity profile with a suitable reference platelet reactivity profile for that individual. The platelet reactivity profile includes one or more of dose response curves and parameters selected from the group comprising EC values, hill slope variability, maximal aggregation values, and minimal aggregation values. The choice of reference profile is determined by the patient; for example, if the patient is undergoing single, dual, or triple anti-platelet therapy, the reference profile will generally be the mean of a cohort of patients undergoing such therapy (an example of a reference profile (consisting of dose response curves only) for normal healthy patients, as well as one for patents with cardiovascular disease undergoing dual anti-platelet therapy is provided below). To identify an individual as having a platelet reactivity profile that is different to that of a reference profile, one or more of the platelet reactivity parameters that form part of the patients platelet reactivity profile are compared with the equivalent parameters in the reference profile. Ideally, the comparison is carried out by comparing the parameter (or parameters) using the Fisher Exact test to ascertain whether the parameter is different in a significant or non-significant manner. A p value is calculated and if the value is less than 0.05 the difference between the parameters is deemed significant (Fisher, R. A. 1922. “On the interpretation of χ² from contingency tables, and the calculation of P”. Journal of the Royal Statistical Society 85(1):87-94, Fisher, R. A. Statistical Methods for research workers. Oliver and Boyd, 1954.). For example, if the patient is a patient with established cardiovascular disease undergoing aspirin therapy, and the epinephrine EC₅₀ values for the patient is higher than that of the reference profile, this would indicate to a clinician that the patient is not adequately inhibited and that a modified ant-platelet therapy is required.

A further means of identifying a platelet reactivity profile that is significantly different (or increased) from that of a reference profile (which method may be used in addition to, or as an alternative to, the methods described above) is to superimpose the patients dose response curves on the equivalent dose response curves from the reference profile, and if they clearly do not superimpose, then the profiles are different. With this method of comparison, if the response is shifted up and to the left, this would indicate that the patients platelets are more reactive compared to the reference, and that the patient may be at risk of suffering an atherothrombotic event.

The invention also relates to a kit suitable for generating a platelet function profile in a rapid, high-throughput, manner, the kit comprising:
a device having a multiplicity of reaction wells;
at least three platelet function modulators; and
instructions for carrying out a method of the invention.

Suitably, the device is a microtitre plate or an equivalent device such as a miniaturised microtitre plate or a cartridge having a multiplicity of reaction vessels.

In one embodiment, the kit is packaged with the platelet function modulators in-situ in the wells of the device.

Preferably, the wells of the device hold a range of concentrations of at least four platelet function modulators, and ideally at least five platelet function modulators. Typically, the device hold at least five, six, seven, or eight concentrations of the platelet function modulator. Ideally, at least one, and preferably two or three, of the agonists is provided in a range of concentrations that includes sub-maximal concentrations for that agonist.

Ideally, the platelet function modulators are platelet agonists, preferably comprising, consisting of, or selected from the group comprising ADP, arachidonic acid, epinephrine, TRAP and collagen. Typically, the platelet function modulators are in liquid form. However, in an alternative embodiment, the platelet function modulators are in a lyophilised form (in which case the platelet function modulators may be stored for weeks or months, optionally stored in the wells of the plate for weeks or months). This is achieved, for example, by adding a solution of the various modulators (i.e. platelet agonists) to the wells of the plate, and then placing the plate in a freeze dryer to lyophilise the agonist solutions, leaving just lyophilised agonist in the wells of the plate. The plates may then be covered by a suitable cover, such as for example, a peel-off top, or capping lids of the type sold for use with microtitre plates.

Thus, in one embodiment, the platelet agonists are lyophilised in-situ in the wells of the microtitre plate.

In one embodiment, the platelet function modifiers are selected from the group comprising: TRAP; collagen; epinephrine; ADP; arachidonic acid; serotonin; thromboxane A2; convulxin; a NO donor (i.e. SNAP); and U46619. Preferably, the platelet function modifiers are selected from the group comprising: TRAP; collagen; epinephrine; ADP; and arachidonic acid.

Ideally, the microtitre plate is a 96 well microtitre plate. As an alternative, a miniaturised, or modified, version of the microtitre plate may be employed.

Generally, the instructions indicate that the platelet function modifier is added to the plate in at least three, four, five, six, seven, eight, or nine concentrations. Typically, arachidonic acid will be provided at concentrations that span the range of 1 to 100 mg/ml, preferably span the range of 3 to 70 mg/ml, and more preferably span the range of 0.58 mg/ml to 50 mg/ml. Typically, collagen will be provided that span the range of 0.001 to 0.3 mg/ml, preferably span the range of 0.0015 to 0.25 mg/ml, and more preferably span the range of 0.0023 to 0.19 mg/ml. Typically, ADP will be provided in concentrations that span the range of 0.01 to 40 μM, preferably 0.2 to 30 μM, and more preferably 0.015 to 20 μM. Typically, epinephrine will be provided in concentrations that span the range of 0.01 to 40 μM, preferably 0.125 to 30 μM, and more preferably 0.0122 to 20 μM. Typically, TRAP will be provided in concentrations that span the range of 0.015 to 20 μM. Generally, the range of concentration employed for the platelet function modulators should span four log units, and should encompass the known effective dose range for each of the platelet function modulators employed.

### BRIEF DESCRIPTION OF THE FIGURES

The invention will be more clearly understood from the following description of some embodiments thereof, given by way of example only, in which:

FIG. 1 shows a mean platelet reactivity profile for normal (healthy) individuals;

FIG. 2 shows a mean platelet reactivity profile for normal (healthy) males and females;

FIG. 3 shows a platelet reactivity profile for an inflamed rheumatoid arthritis patient;

FIG. 4 shows a platelet reactivity profile was obtained for a 37 year old male with coronary thrombosis undergoing an anti-platelet therapy of ASPIRIN, CLOPIDOGREL and ABCIXIMAB;

FIG. 5a shows a dose response curve for the agonist epinephrine generated using conventional serial dilution concentrations of epinephrine;

FIG. 5b shows a dose response cure for epinephrine generated using a range of concentrations that include sub-maximal concentrations for epinephrine;

FIG. 6 (FIGS. 6a to 6e) shows a mean platelet reactivity profile for 50 normal (healthy) volunteers (A) versus a mean profile for 75 patients with known cardiovascular disease (o) who are taking dual anti-platelet therapy consisting of ASPIRIN 75 mg and CLOPIDOGREL 75 mg; and

FIG. 7 (FIGS. 7a to 7e) shows a mean platelet reactivity profile for 75 patients with known cardiovascular disease who are taking dual anti-platelet therapy consisting of ASPIRIN 75 mg and CLOPIDOGREL 75 mg (∘) versus an individual male who has previously had two myocardial infacrtions and a further thrombotic stroke (▴=visit 1, □=visit 2).

### DETAILED DESCRIPTION OF THE INVENTION

Materials and Methods

Healthy subjects who had not taken any antiplatelet medication in the 14 days prior to the study were recruited. Subjects refrained from intensive exercise and tobacco use for 4 hours prior to early morning phlebotomy. Blood was drawn from the antecubital fossa. The first 5 ml taken was discarded to avoid unwanted platelet activation and the next 27 ml of blood was collected in 3 ml of 3.2% sodium citrate anticoagulant. This blood citrate mixture was then centrifuged at 150 g for 10 min to recover the supernatant platelet rich plasma (PRP). The platelet concentration was measured using a Sysmex KX 21N.

96 Well Plate Preparation:

A 96 well black isoplate with clear bottoms was used. Agonists were arranged in rows of Row 1—Arachidonic Acid (AA), Row 2—Collagen, Row 3—ADP, Row 4—Epinephrine, Row 5—Thrombin related activated peptide (TRAP) and Row 6 as a control row containing 4 wells of PRP and 4 wells of Platelet Poor Plasma (PPP). The remaining 6 rows maybe used for additional agonists or specific peptides as required.

Buffer A (6 mM Dextrose, 130 mM NaCl, 9 mM NaHCO₃, 10 mM trisodium citrate, 10 mM Tris Base, 3 mM KCl, 0.81 mM KH₂PO₄, and 0.9 mM MgCl₂6H₂O [pH7.4]) was added to wells 2-8 on each row using a 12 channel pipette. Row 1—10 μl of buffer A was inserted into well 2. Then 20 μl of buffer A was added to each of the wells 3-8 inclusive. Then 50 μl of AA (500 mg/ml stock) was inserted into well 1. 30 μl of AA was removed from well 1 and mixed with buffer A in well 2. 20 μl of the AA/buffer A was removed from well 2 and mixed in well 3. 20 μl is then removed from well 3 and mixed in well 4. This is repeated throughout the row up to and including well 8 with 20 μl left over and discarded. Row 2—Collagen (1.9 mg.ml stock) same process as Row 1. Row 3 & 5 ADP (Row 3) and TRAP (Row 5), wells 2-8 had 20 μl of buffer A placed into each. A quantity of 40 μl of ADP (200 μM stock) was placed into well 1. 20 μl of agonist was removed from well 1 and placed into well 2 where it was then mixed thoroughly with buffer A. This same process was then repeated across the plate with 20 μl left over after well 8. This was then discarded. The same process is repeated for Row 5—TRAP (200 μM stock). Row 4 Epinephrine—Using a separate 96 well mixing plate 20 μl of buffer A is added to wells 2-8 and to a second row wells 1-8 to give 15 wells containing buffer A. The process of serial dilution is repeated as with agonists ADP and TRAP with 16 concentrations made up instead of 8. The first well has 40 μl of Epinephrine (200 μM stock) added to well 1. The agonist/buffer mixture is removed from the mixing plate wells 1, 3, 5, 7, 9, 11, 13, 15 and is then transferred to the master plate. This method is adapted to make maximum 8 plates at a time.

Once the plate is prepared the PRP is added. 180 μl of PRP is added to each row using a multi-channel pipette and reverse pipetting technique is used to avoid any bubble formation within the well. This gives a final volume of 200 μl.

The plate is then placed into a Wallac Victor 3 plate reader. The plate is read at time zero (T0) at absorbance 572 nm. The plate is then set to shake at 1000 rpm on an orbit of 0.1 mm for 3 minutes. T3 (3 minutes) read is taken and then shaking recommences until T9 (9 minutes) read. Further reads are taken at T15 (15 minutes) and T18 (18 minutes) with shaking in between each time-point. The entire protocol is performed at 37° C.

The data is then normalised from the PPP and PRP absorbance values which represent minimum and maximum aggregation. The data is then inserted into PRISM software and analysis performed to calculate maximal aggregation, EC50 and hillslope variability.

### Example 1

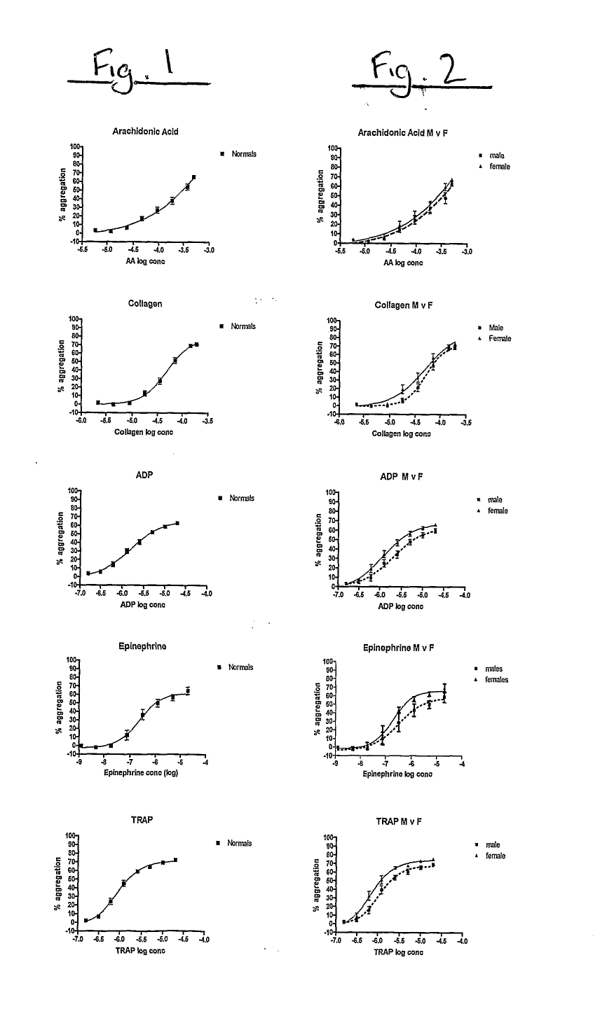

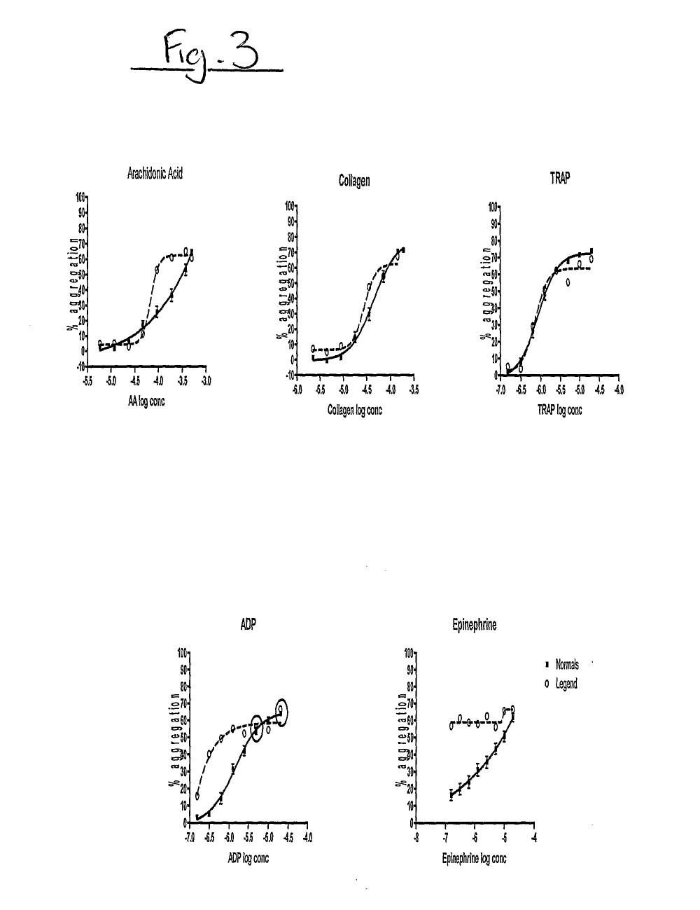

The Materials and Methods above were employed to generate platelet reactivity profiles of 50 healthy males and females. FIG. 1 (FIGS. 1a to 1e) shows an average platelet reactivity profile for healthy males, and FIG. 2 (FIGS. 2a to 2e) shows an average platelet reactivity profile for healthy females. What this clearly demonstrates is the value of using multiple concentrations of multiple agonists. You can see that if a single traditional concentration of a particular agonist is used (represented by the concentration at which maximal aggregation occurs on the far right of each graph) we do not pick up on biological variation. This is clearly seen at submaximal concentrations by the significant difference in platelet reactivity between males and females at these specially formulated concentrations.

### Example 2

A platelet reactivity profile for an inflamed rheumatoid arthritis patients was generated—See FIG. 3 (FIGS. 3a to 3e). The Rheumatoid Arthritis population are an inflamed group who have increased thrombotic risk. Our assay clearly demonstrated excessive platelet reactivity from agonists Arachidonic Acid (FIG. 3a) ADP (FIG. 3d) and Epinephrine (FIG. 3e) in this inflamed rheumatoid arthritis patient. Using the methods described by Gerber, the use of ADP5 and ADP20 would have missed this major clinical problem. This is highlighted in FIG. 3d with the two circles identifying ADP5 μM and 20 μM compared to the response expected to be seen in the normal population (n=50). The fact that this reactivity occurs in some but not all of the agonists used as standard in this assay points towards a specific mechanism for this profound hyperreactivity. This example also highlights the absolute value of utilising submaximal concentrations of multiple agonists.

### Example 3

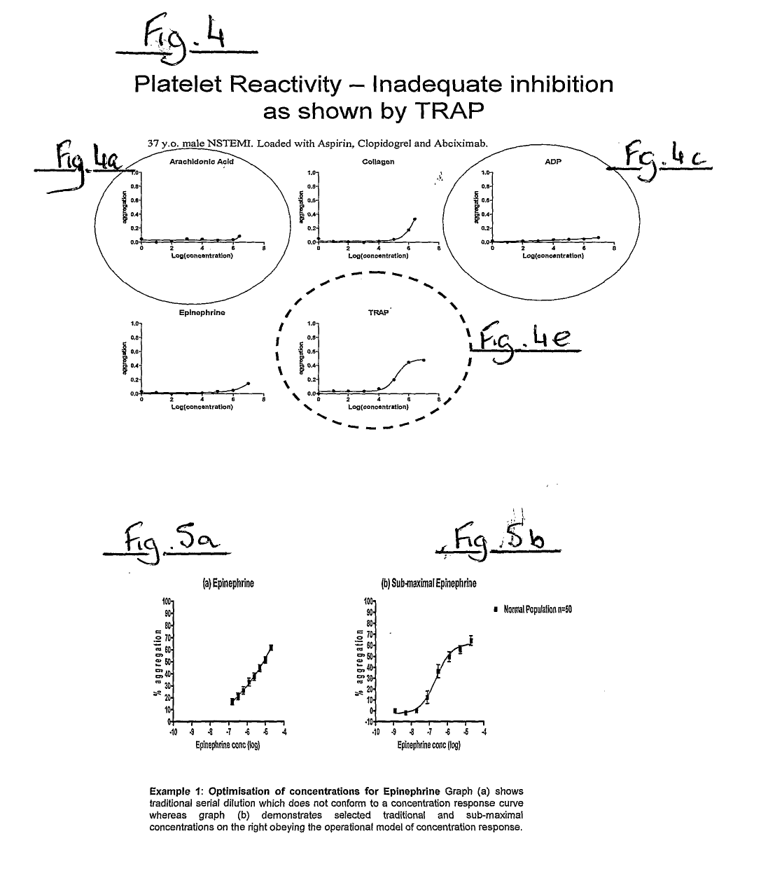

A platelet reactivity profile was obtained for a 37 year old male with coronary thrombosis undergoing an anti-platelet therapy of ASPIRIN, CLOPIDOGREL and ABCIXIMAB, and is shown in FIG. 4 (FIGS. 4a to 4e). This example highlights that if a platelet reactivity profile was limited to arachidonic acid and ADP aggregation (FIGS. 4a and 4c) at one or two concentration, as per the prior art methods of generating platelet reactivity profiles, the patient would be informed that their antiplatelet therapy is working satisfactorily and that their thrombotic risk is low, when in fact the TRAP dose response curve (FIG. 4e) clearly shows that the antiplatelet regime has not provided acceptable inhibition in the individual and that their risk of having a future atherothrombotic event remains significant.

### Example 4

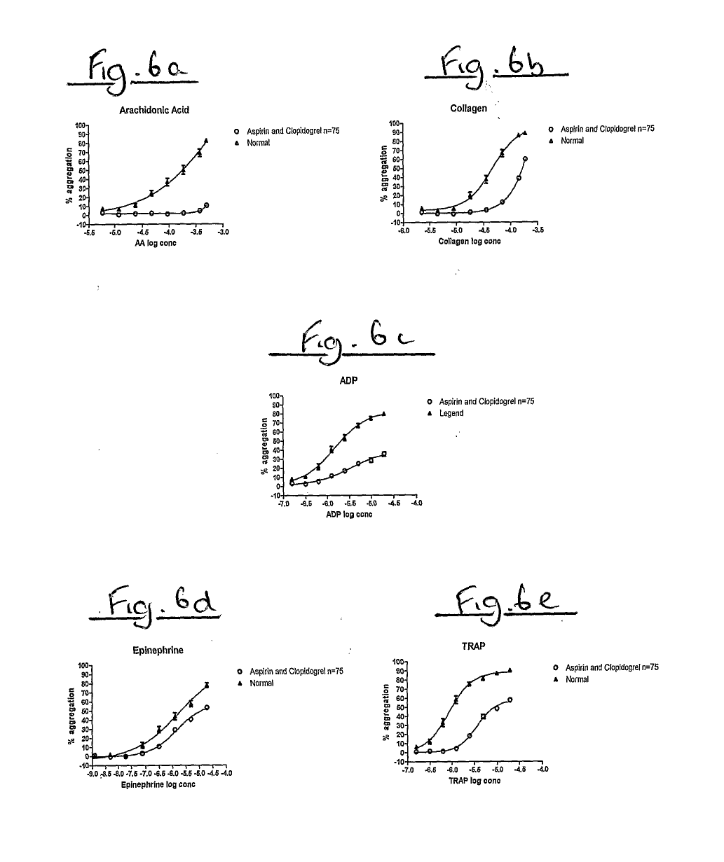

FIG. 5a shows a dose response curve for the agonist epinephrine generated using conventional serial dilution concentrations of ephinephrine. FIG. 5b shows a dose response cure for epinephrine generated using a range of concentrations that include sub-maximal concentrations for epinephrine. Clearly, FIG. 5a does not respond to a concentration response curve, whereas FIG. 5b obeys the operational model of concentration response.

### Example 5

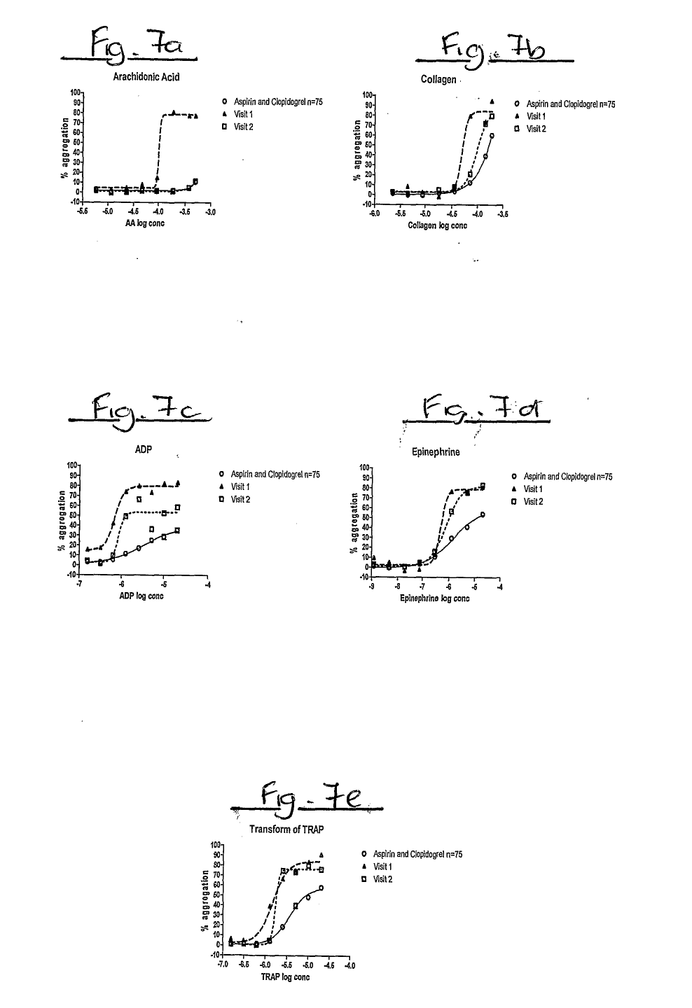

FIG. 6 (FIGS. 6a to 6e) shows a mean platelet reactivity profile for 50 normal (healthy) volunteers (A) versus a mean profile for 75 patients with known cardiovascular disease (∘) who are taking dual anti-platelet therapy consisting of ASPIRIN 75 mg and CLOPIDOGREL 75 mg.

### Example 6

FIG. 7 (FIGS. 7a to 7e) shows a mean platelet reactivity profile for 75 patients with known cardiovascular disease who are taking dual anti-platelet therapy consisting of ASPIRIN 75 mg and CLOPIDOGREL 75 mg (∘) versus an individual male who has previously had two myocardial infacrtions and a further thrombotic stroke (▴=visit 1, □=visit 2).

The above set of figures clearly demonstrates a man who has demonstrated increased cardiovascular risk by previously having 2 myocardial infarctions and a further thrombotic stroke. He is taking an enteric coated aspirin 75 mg daily and clopidogrel 75 mg daily at the time of visit one and his platelet reactivity is tested. This demonstrates a hyperresponsive phenotype across the 5 tested agonists with little or no platelet inhibition from his current antiplatelet regime. The patient returns 4 weeks later and in the meantime has had his medication changed to aspirin (soluble) 150 mg and clopidogrel remains as before at 75 mg once daily. If we examine the Arachidonic acid graph and now focus on visit 2 we see that the patient's COX pathway which aspirin is responsible for antagonising is clearly inhibited and is comparable to the results seen in 75 other individuals on dual antiplatelet therapy. There has been a consistent fall in his platelet reactivity; however despite this therapy the assay demonstrates its value in that the other agonists pick up that the thrombotic risk of the patient remains significantly higher despite treatment

This highlights that
1. the assay clearly identifies those at risk of thrombotic events
2. that the change in clinical management has resulted in inhibition of one pathway responsible for thrombosis
3. but despite the decrease in platelet reactivity he remains to have platelet reactivity that is significantly higher than the rest of the cardiovascular population on dual aspirin and clopidogrel despite being on a higher dose.

### Example 7

The Applicant has employed the methods of the invention in investigating the platelet activity modulating ability of synthetic peptides as described in the following paper: Edwards, R. J., et al., Bioinformatic discovery of novel bioactive peptides. Nat Chem Biol, 2007. 3(2): p. 108-12.

The invention is not limited to the embodiments hereinbefore described which may be varied in both construction and detail without departing from the spirit of the invention.

1. A method of determining a platelet reactivity profile of an individual comprising the steps of:
providing a platelet-containing biological sample from the individual;
providing at least three platelet function modulators, each platelet function modulator being provided in at least three concentrations;
reacting an aliquot of the platelet containing sample with each concentration of each platelet function modulator in a separate reaction vessel;
measuring platelet aggregation in each reaction vessel; and
using the platelet aggregation measurements to generate a dose response curve for each platelet function modulator, wherein the dose response curves obtained and/or one or more functions of the dose response curves obtained, comprise a platelet reactivity profile for the individual.

2. A method as claimed in claim 1 in which at least four platelet function modulators are employed.

3. A method as claimed in claim 1 in which at least five platelet function modulators are employed.

4-37. (canceled)

38. A method as claimed in claim 1 in which the platelet function modulators are platelet agonists.

39. A method as claimed in claim 3 in which at least five platelet agonists are employed, the agonists comprising TRAP, collagen, epinephrine, ADP, and arachidonic acid.

40. A method as claimed in claim 1 in which the function(s) of the dose response curve is selected from one or more of hill slope variability, maximum and/or minimum aggregarion, and EC values (i.e. EC50).

41. A method as claimed in claim 1 in which the reaction vessels are wells of a microtitre plate or an equivalent device having a multiplicity of reaction wells.

42. A method as claimed in claim 1 which is a high-throughput method in which the parameter of platelet aggregation in each reaction vessel is measured substantially simultaneously.

43. A method as claimed in claim 1 in which each platelet function modulator is provided in a range of concentrations spanning at least two log units.

44. A method as claimed in claim 1 in which the platelet function modulators are platelet agonists, and wherein at least one of the platelet agonists is employed in a concentration range that includes sub-maximal concentrations for that agonist.

45. A method as claimed in claim 44 in which the at least one platelet agonists that is employed in a concentration range that includes sub-maximal concentrations for that agonist is one or more of collagen, epinephrine, and arachidonic acid.

46. A method of determining the platelet reactivity status of an individual which comprises the step of determining the platelet reactivity profile for the individual according to a method claim 1, and comparing the platelet reactivity profile obtained with a reference platelet reactivity profile for that individual.

47. A method of identifying an individual at risk of having an atherothrombotic event, which method comprises the steps of determining the platelet reactivity profile for an individual according to a method of claim 1, and comparing the platelet reactivity profile obtained with a reference platelet reactivity profile for that individual, wherein if the platelet reactivity profile for the individual shows a significantly increased response to an agonist as compared to the response to that agonist in the reference platelet reactivity profile, then that individual is at risk of having an atherothrombotic event.

48. A method of identifying aberrant platelet reactivity in an individual, which method comprises the steps of determining the platelet reactivity profile for an individual according to a method of claim 1, and comparing the platelet reactivity profile obtained with a reference platelet reactivity profile for that individual, wherein if the platelet reactivity profile for the individual is significantly different to the reference platelet reactivity profile, then that individual has aberrant platelet reactivity.

49. A method of identifying a suitable anti-platelet agent or dose for an individual in need thereof, which method comprises the steps of determining the platelet reactivity profile for the individual according to a method of claim 1, comparing the platelet reactivity profile obtained with a reference platelet reactivity profile for that individual, identifying any agonist for which there is a significantly increased response when compared to the response to that agonist in the reference platelet reactivity profile, and choosing an anti-platelet therapy or dose to target the biological pathway modulated by that agonist.

50. A method of identifying and correcting inadequate or sub-optimal anti-platelet therapy in an individual, which method comprises the steps of determining the platelet reactivity profile for the individual according to a method of claim 1, comparing the platelet reactivity profile obtained with a reference platelet reactivity profile for an individual undergoing the anti-platelet therapy, identifying any agonist which is inadequately inhibited compared with the reference profile, and modifying the anti-platelet therapy to effect adequate inhibition of the agonist.

51. A method as claimed in claim 50 in which modification of the therapy involves changing the drugs employed, or changing the dosage regime for that drug.

52. A method of identifying platelet activity modulating agents, the method comprising the step of determining the platelet reactivity profile for an individual according to a method of claim 1 in the absence and presence of a test compound, comparing the platelet reactivity profiles obtained, and where there is a significant difference between the platelet reactivity profiles obtained, determining whether the test compound is a platelet agonist or a platelet antagonist.

53. A method as claimed in claim 52 in which a platelet reactivity profile is obtained for a number of different concentrations of the test compound.

54. A method of screening a library of test compounds for platelet activity modulating agents, which method employs a method of identifying platelet activity modulating agents according to claim 52.
